(19) United States
(12) Patent Application Publication
(10) Pub. No.: US 2024/0207352 A1
(43) Pub. Date: Jun. 27, 2024

(54) COMPOSITIONS AND METHODS FOR MODULATING MAMMARY DISORDERS AND CONDITIONS

(71) Applicant: MILEUTIS LTD., Ness Ziona (IL)

(72) Inventors: Jose Mario ISCOVICH, Gan Yavne (IL); Javier ISCOVICH, Tel Aviv (IL)

(73) Assignee: MILEUTIS LTD., Ness Ziona (IL)

(21) Appl. No.: 18/557,393

(22) PCT Filed: Apr. 28, 2022
(86) PCT No.: PCT/IL2022/050442

§ 371 Date: Oct. 26, 2023

Related U.S. Application Data
(60) Provisional application No. 63/180,727, filed on Apr. 28, 2021.

(51) Int. Cl. A61K 38/01; A61K 9/00; A61K 33/245; A61K 38/07; A61K 38/08; A61K 38/10; A61K 38/17; A61K 47/44

(52) U.S. Cl. CPC A61K 38/018; A61K 9/0041; A61K 33/245; A61K 38/07; A61K 38/08; A61K 38/10; A61K 38/1709; A61K 47/44

(57) ABSTRACT

The present disclosure provides compositions and methods for modulating various mammary' disorders and conditions. The disclosure further provides a therapeutic combination of a composition of at least one casein protein or casein-derived peptide; and a teat seal composition.

### SEQUENCE LISTING STATEMENT

The instant application contains a Sequence Listing which has been submitted electronically in ASCII format and is hereby incorporated by reference in its entirety. Said ASCII copy, created on Apr. 26, 2021, is named P-596301-USP_SL.txt and is 13,632 bytes in size.

### BACKGROUND OF THE INVENTION

Bovine mastitis is a persistent, inflammation of a mammary gland or an udder caused by a variety of causes, such as physical injury, introduction of chemicals, viruses, fungus, parasites or, most commonly, bacterial invasion and their toxins with considerable implications to dairy cattle worldwide.

Casein-derived peptides were previously reported in the treatment of mammary gland infection. In addition, physical barrier devices such as teat seal and hydrogel formulations were described, infused alone, or in combination with analgesics, antimicrobials, or anti-inflammatory drugs, separately or blended, into the teat canal or to the teat sinus or teat surface.

There remains a need for improved measures to prevent and treat mammary disorders and conditions, such as mastitis.

### SUMMARY OF THE INVENTION

The present invention provides, in one aspect, a therapeutic combination, comprising: a composition comprising at least one casein protein or casein-derived peptide; and a teat seal composition.

In certain embodiments, the at least one casein protein comprises at least one of β-casein, αS1-casein, (S2-casein, or K-casein.

In certain embodiments, the at least one casein-derived peptide comprises at least one fragment of β-casein, αS1-casein, αS2-casein, or K-casein.

In certain embodiments, the at least one casein-derived peptide comprises a casein hydrolysate.

In certain embodiments, the at least one casein-derived peptide comprises a phosphopeptide. In certain embodiments, the phosphopeptide comprises an amino acid sequence selected from the group consisting of SEQ ID NO:1 to SEQ ID NO:26.

In certain embodiments, the teat seal composition forms a physical barrier on a teat surface, in a teat canal, or in a teat cistern of an animal.

In certain embodiments, the teat seal composition is formulated as a dip, a hydrogel, or a biofilm.

In certain embodiments, the teat seal composition comprises a heavy metal, or salt thereof. In certain embodiments, the teat seal composition comprises a non-toxic heavy metal salt. In certain embodiments, the non-toxic heavy metal salt is selected from the group consisting of bismuth salt, titanium salt, zinc salt, barium salt, and any combination thereof.

In certain embodiments, the teat seal composition comprises 40% to 80% by weight heavy metal salt.

In certain embodiments, the casein-derived peptide composition comprises between 10 ng/ml to 500 mg/ml of the at least one casein-derived peptide.

In certain embodiments, the at least one casein protein or casein-derived peptide composition and the teat seal composition are comprised in the same composition.

In certain embodiments, the at least one casein protein or casein-derived peptide composition and the teat seal composition are comprised in different compositions.

In certain embodiments, the at least one casein protein or casein-derived peptide composition and the teat seal composition are administrated separately.

In certain embodiments, the at least one casein protein or casein-derived peptide composition and the teat seal composition are administrated together.

In certain embodiments, the at least one casein protein or casein-derived peptide composition or the teat seal composition further comprise a pharmaceutical carrier.

In certain embodiments, the pH of the at least one casein protein or casein-derived peptide composition or the teat seal composition is in the range of between about 6.5 to about 9.0.

The present invention further provides, in another aspect, a method for: treating, preventing, inhibiting, reducing, eliminating, protecting or delaying the onset of a mammary gland disorder; reducing the length of the dry period; increasing the milk yield; increasing the milk hygiene; increasing the cow's comfort; improving the livestock welfare, enhancing involution, increasing the rate of involution; or any combination thereof; in a female mammalian animal in need thereof, the method comprising administering a therapeutic combination, comprising (i) a composition comprising at least one casein protein or at least one casein-derived peptide; and (ii) a teat seal composition; to the female mammalian animal.

In certain embodiments, the therapeutic combination is administrated at the lactating period of the female mammalian animal.

In certain embodiments, the therapeutic combination is administrated at the dry period of the female mammalian animal.

In certain embodiments, the at least one casein protein or the at least one casein-derived peptide composition is administered one to six times.

In certain embodiments, the at least one casein protein or the at least one casein-derived peptide composition is administered at intervals of from about 1 hour to about 24 hours.

In certain embodiments, the teat seal composition is administered during the administration of the at least one casein protein or the at least one casein-derived peptide composition.

In certain embodiments, the teat seal composition is administered after the administration of the at least one casein protein or the at least one casein-derived peptide composition.

In certain embodiments, the teat seal composition and the at least one casein protein or the at least one casein-derived peptide composition are sequentially administrated, one immediately after the other.

In certain embodiments, the teat seal composition and the at least one casein protein or the at least one casein-derived peptide composition are sequentially administrated. at about 1 to about 24 hours intervals.

In certain embodiments, the teat seal composition and the at least one casein protein or the at least one casein-derived peptide composition are sequentially administrated at about 1 to about 48 hours intervals.

In certain embodiments, the teat seal composition and the at least one casein protein or the at least one casein-derived peptide composition are sequentially administrated at about 24 hours intervals. In certain embodiments, the teat seal composition and the at least one casein protein or the at least one casein-derived peptide composition are sequentially administrated at about 48 hours intervals. In certain embodiments, the teat seal composition and the at least one casein protein or the at least one casein-derived peptide composition are sequentially administrated at about 72 hours intervals.

In certain embodiments, the teat seal composition is administered up to 12 hours after the administration of the at least one casein protein or the at least one casein-derived peptide composition.

In certain embodiments, the at least one casein protein or the at least one casein-derived peptide composition and the teat seal composition are administrated to at least one mammary gland of the female mammalian animal.

In certain embodiments, the at least one casein protein or the at least one casein-derived peptide composition and the teat seal composition are administrated to at least one udder of the female mammalian animal.

In certain embodiments, the at least one casein protein or the at least one casein-derived peptide composition and the teat seal composition are administrated to at least one teat canal of the female mammalian animal.

In certain embodiments, the mammary gland disorder is a mammary gland infection.

In certain embodiments, the mammary gland infection is mastitis. In certain embodiments, the mastitis is selected from the group consisting of clinical mastitis, subclinical mastitis, and chronic mastitis.

In certain embodiments, increasing the milk hygiene comprises preventing black spot defect (BSD) in cheese, reducing somatic cell counts (SCC) in milk or combination thereof. In certain embodiments, increasing the milk hygiene comprises preventing black spot defect (BSD) in cheese. In certain embodiments, increasing the milk hygiene comprises reducing somatic cell counts (SCC) in milk.

In certain embodiments, the female mammalian animal is a lactating animal. In certain embodiments, the female mammalian animal is a non-lactating animal. In certain embodiments, the female mammalian animal is selected from the group consisting of a cow, a goat, a sheep, a buffalo, a camel, a donkey, a llama, a horse, a pig, a cat, and a dog. In certain embodiments, the female mammalian animal is a cow.

In certain embodiments, the method comprises administering between 10 ng to 200 mg of the at least one casein protein or casein-derived peptide per kg-body-weight of the female mammalian animal.

The present invention further provides, in another aspect, a kit comprising (i) a composition comprising at least one casein protein or at least one casein-derived peptide, (ii) a teat seal composition, and optionally (iii) instructions for use of the kit in a method for: treating, preventing, inhibiting, reducing, eliminating, protecting or delaying the onset of a mammary gland disorder; reducing the length of the dry period; increasing the milk yield; increasing the milk hygiene; increasing the cow's comfort; improving the livestock welfare, enhancing involution, increasing the rate of involution; or any combination thereof.

In certain embodiments, the kit comprises at least two separate containers, first container comprising the composition comprising the at least one casein protein or at least one casein-derived peptide, and the second container comprising the teat seal composition.

In certain embodiments, the first container or the second container are a syringe.

In certain embodiments, the kit comprises at least one container, the container comprising both the composition comprising the at least one casein protein or at least one casein-derived peptide and the teat seal composition.

In certain embodiments, the container is a syringe.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present methods, compositions and methods of use thereof may be understood more readily by reference to the following detailed description which forms a part of this disclosure. It is to be understood that this disclosure is not limited to the specific products, methods, conditions or parameters described and/or shown herein, and that the terminology used herein is for the purpose of describing particular embodiments by way of example only and is not intended to be limiting of the claimed disclosure. Similarly, it is to be understood that the embodiments disclosed herein are combinable.

Mammary gland infections of mammalian animals possess difficulties in milk production including reduced milk production and milk quality. Common treatments such as antimicrobial agents (e.g., antibiotics, antiseptics) are often associated with development of bacterial resistance.

The inventors found that the combined administration of teat seal compositions and casein proteins and peptides is possible during the pre-partum period of the animal without worsening the milk quality or milk products, and during the postpartum period of the animal. In addition, the inventors have found that administration of the casein proteins and peptides and the teat seal composition results in an improvement of at least one condition associated with the mammary gland, such as mastitis.

Further, it was found that the therapeutic combination reduced the levels of at least one metal in the milk and milk products.

Thus, in accordance with first aspect, the present disclosure provides a therapeutic combination comprising at least one casein protein or peptide and a teat seal composition. The combination described herein is at times referred herein to a veterinary combination.

Casein is a protein in non-human mammal's milk, also found in human mammal's milk known to include the subgroups αS1, αS2, β and κ. Casein is defined according to the amino acid sequences of each of the subgroups αS1, αS2, β and κ. In the context of the present disclosure, when referring to casein, it is to be understood as also including acid casein, salts of casein, phosphorous containing casein and rennet casein.

The term “peptide” as used herein refers to amino acid residues, connected by peptide bonds. A peptide sequence is generally reported from the N-terminal end containing free amino group to the C-terminal end containing free carboxyl group. Amino acids, as used herein, refer to naturally occurring and synthetic amino acids, as well as amino acid analogs, and amino acid mimetics, that function in a manner similar to the naturally occurring amino acids. Amino acids may be referred to herein by either their commonly known three letter symbols or by the one-letter symbols recommended by the IUPAC-IUB Biochemical Nomenclature Commission.

The casein-derived peptide may be a single peptide or a mixture of different peptides which may be independently selected from a naturally occurring peptide (casein protein), a semi-synthetic peptide, a synthetic peptide or a recombinant peptide. It should be further noted that the peptides according to the present disclosure may be produced synthetically, or by recombinant DNA technology, or by any other technology. Methods for producing peptides are well known in the art.

In some embodiments, the casein-derived peptide may comprise a casein protein breakdown product which occurs when casein protein is cleaved by enzymes or acids to peptide fragments (also known in the art by the term “casein hydrolysate”). A casein hydrolysate is to be understood as the hydrolyzed form of casein (protein). Casein hydrolysate includes, for example, the active beta-, alpha S1-, alpha S2-, kappa-casein-derived peptide known to those versed in the art. In some embodiments, the casein-derived peptide is or comprises a casein hydrolysate.

Naturally-occurring casein-derived peptides are typically obtained following enzymatic hydrolysis, the enzyme may be any mammal peptidase, such as, without being limited thereto, plasmin, pancreatin, trypsin, chymotrypsin, neutrase, alcalase, pepsin, carboxypeptidase, cathepsin, as well as plant peptidase such as, without being limited thereto, papain, bromelain, as well as enzymes from microorganism source. For example, a naturally occurring casein-derived peptide may be the result of an enzyme activity such as plasmin on casein subunits β-casein, αs1- and αs2-casein or x-casein. In some embodiments, a casein hydrolysate is obtained by cleavage of the casein protein with trypsin.

In some embodiments, the casein-derived peptide is a synthetic, semi synthetic or a recombinant peptide. In one embodiment, the casein-derived peptide is a synthetic peptide. In another embodiment, the casein-derived peptide is a semi synthetic peptide. In another embodiment, the casein-derived peptide is a recombinant peptide.

A synthetic peptide may be obtained by any methods known in the art of peptide synthesis including chemical synthesis and recombinant DNA technology. For example, the peptides may be synthesized by using standard solid phase techniques.

A semi-synthetic casein-derived peptide may be obtained by chemical hydrolysis of casein, e.g. by prolonged boiling in a strong acid (acid-HVP) or strong base or using a chemical agent such as Cyanogen bromide (CNBr). The casein-derived peptide may also be obtained by molecular engineering, e.g. using recombinant DNA, in molecular techniques known in the art. In such embodiment, the casein-derived peptide is a recombinant peptide.

In some embodiments, the casein-derived peptide may be a phosphopeptide. As used herein, the term “phosphopeptide” designates a phosphorylated peptide in form of a conjugated peptide in which the non-peptide portion is a residue of phosphoric acid. The expression “phosphopeptide” or “phosphoserine” designates conjugated serine in which the non-peptide portion is a residue of phosphoric acid.

In some embodiments, the casein-derived peptide is a single peptide or mixture of a phosphopeptide, namely, which contains a single phosphorous group or is a phosphorus-enriched peptide. In some embodiment, the casein-derived peptide is any phosphoserine, phosphotyrosine, phosphothreonine, and/or phosphohystidine-enriched casein-derived peptides (casein phosphopeptide, CPP) and monovalent cation phosphocaseinates, such as sodium, potassium, calcium or ammonium phosphocaseinates.

In some embodiment, the casein-derived peptide enriched with phosphorous groups is a casein hydrolysate.

In some embodiment, the casein-derived peptide enriched with phosphorous groups is a synthetic or semi-synthetic peptide.

In some embodiment, the casein-derived peptide enriched with phosphorous groups is a recombinant peptide.

In some embodiments, the casein-derived peptide is a phosphor-peptide.

The phosphor-peptide may be genetically engineered casein-derived peptides as well as peptidomimetics of casein-derived peptides. For example, phosphorylation of amino acids such as at least one serine residue may be performed by any method as is known in the art. The term “casein-derived peptide” also encompasses peptide fragments or peptidomimetic products obtained from or corresponding to one or more sections of casein protein. The peptidomimetic peptide may be for example a peptoid or a semipeptoid, which are peptide analogs, having, for example, modifications such as, but are not limited to, cyclization, N-terminus modification, C-terminus modification, peptide bond modification, including, but not limited to, CH₂—NH, CH₂—S, CH₂—S—O, O—C—NH, CH₂—O, CH₂—CH₂, S—C—NH, CH—CH or CF—CH, backbone modification and residue modification.

As used herein, the term “casein-derived peptide” further encompasses any derivatives, analogues, variants or homologues of any of the peptides. The term “derivative” is used to define amino acid sequences (peptide), with any insertions, deletions, substitutions and modifications to the amino acid sequences (peptide) that do not alter the activity of the original peptides. By the term “derivative” it is also referred to homologues, variants and analogues thereof, as well as covalent modifications of a polypeptides made according to the present invention.

In some embodiments, the modified, synthetic, semi-synthetic or other types of analogs of the naturally occurring casein-derived peptides are at least 75%, at times 85%, 90%, 95% and even 99% identical (in sequence) to a naturally occurring casein-derived peptide when the two sequences are optimally aligned. Further, any non-naturally occurring casein-derived peptide to be used in accordance with the present disclosure may retain at least part of the biological activity of the naturally occurring casein protein (e.g. treating, enhancing, preventing, inhibiting, reducing, eliminating, protecting, improving or delaying the onset of a condition associated with the mammary gland in a female mammalian animal).

The present disclosure also encompasses homologues of the casein-derived peptide. The term “homologues” is used to define amino acid sequences (peptide) which maintain a minimal homology to the amino acid sequences defined by the invention, e.g. have at least about 65%, at least about 75%, at least about 85%, or at least about 95% overall sequence homology with the amino acid sequence of any of the peptide as structurally defined above, e.g. of a specified sequence.

In some embodiments, the casein-derived peptide may also include a chemical modification of a naturally occurring peptide, e.g. where one or more amino acids are deleted, substituted or modified, e.g. by removal of a side group, substitution of a side group or the introduction of a chemical group. Without being limited thereto, the chemical modification may include acetylation, acylation, amidation, ADP-ribosylation, glycosylation, GPI anchor formation, covalent attachment of a lipid or lipid derivative, methylation, myristoylation, pegylation, prenylation, phosphorylation, ubiquitination, or any similar process. When referring to replacement of an amino acid sequence by another, it is likely that the replacement is a conservative substitution. For example, one or more amino acid residues within a casein sequence is substituted by another amino acid of a similar polarity or charge. For example, the non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Nonetheless, non-conservative substitutions may also take place as long as it does not significantly change the desired (casein like) biological activity of the resulting casein-derived peptide analog.

A casein-derived peptide in accordance with the present disclosure is characterized by a molecular weight of between about an average 100 to an average 10,000 Dalton (e.g. between 2 to 100 amino acids) at times between about an average 100 to an average 7,000 Dalton and at times between an average 1,000 to an average 5,000 Daltons.

A casein-derived peptide in accordance with the disclosure is characterized by a length of from 2 to 200, from 2 to 100 amino acids, at times between 4 amino acids to 40 amino acids, at times from 4 amino acids to 30 amino acids, at times 4 amino acids to 10 amino acids, at times between 10 amino acids to 50 amino acids.

In some embodiments, the casein-derived peptide comprises an amino acid sequence denoted as Ser-Ser-Ser-Glu (SEQ ID NO:1), wherein at least one Ser residue, at least two Ser residues or three Ser residues are phosphorylated (phosphorylated serine is denoted herein as Ser(p) or S(p)).

In some other embodiments, the casein-derived peptide comprises an amino acid sequence provided by Ser-Ser-Ser-Glu-Glu (SEQ ID NO:2), wherein at least one Ser residue, at least two Ser residues or three Ser residues are phosphorylated.

In some other embodiments, the casein-derived peptide comprises an amino acid sequence provided by Ser(p)-Ser(p)-Ser(p)-Glu-Glu (SEQ ID NO:3).

In some embodiments, the casein-derived peptide comprise at least one sequence provided by: SEQ ID NO:4—RELEELNVPGEIVES(p)LS(p)S(p)S(p)EESITR; SEQ ID NO:5—QMEAESIS(p)S(p)S(p)EEIVPDSVEQK; SEQ ID NO:6—KNTMEHVS(p)S(p)S(p)EES[ISNETYK; SEQ ID NO:7—KVNELSKNIGS(p)ES(p)TEDQ; SEQ ID NO:8—PTLNREQLS(p)TS(p)EENSKKTVD; SEQ ID NO:9—ELEELNVPGEIVES(p)LS(p)S(p)S(p)EESITR.

In some other embodiments, the casein-derived peptide comprises at least one of: amino acids 1-25 in P-casein protein SEQ ID NO:10—RELEELNVPGEIES(p)LS(p)S(p)S(p)EESITR; amino acids 59-79 in αS1-casein protein SEQ ID NO: 1—QMEAES(p)IS(p)S(p)S(p)EEIVPNS(p)VEQK; amino acids 1-21 in αS2-casein protein SEQ ID NO:12—KNTMEHVS(p)S(p)S(p)EESIIS(p)QETYK; amino acids 36-52 in αS1-casein protein SEQ ID NO:13—KVNELSKDIGS(p)ES(p)TEDQ: amino acids 12-140 in αS2-casein protein SEQ ID NO:14—ESIIS(p)QETYKQEKNMAINPSKENLCSTFCKEVVRNANEEETSIGS(p)S(p)S(p)EE S(p)AEVATEEVKITVDDKHYQKALNEINQFYQKFPGYLQYLYQGPIVLNPWNQ VLRNAVPITPTLNREQLS(p)TS(p)EENSKKTVN; amino acids 2-25 in β-casein protein SEQ ID NO:15—ELEELNVPGEIES(p)LS(p)S(p)S(p)EESITR.

In some other embodiments, the casein-derived peptide comprises the amino acids provided by: X₁₍ₙ₎-Ser(p)-Ser(p)-Ser(p)-X₂₍ₘ₎ (SEQ ID NO:16), wherein at least one of X₁ and X₂ is independently selected from a positively charged amino acid and wherein each one of n and m is independently selected from 0, 1 and 2.

In some embodiments, the positively charged amino acid is selected from the group consisting of lysine, arginine and histidine. In some embodiments, the positively charged amino acid is lysine. In some other embodiments, the positively charged amino acid is arginine. In some other embodiments, the positively charged amino acid is histidine.

According to some embodiments, the casein-derived peptide comprises an amino acid sequence selected from the group consisting of: Lys-Lys-Ser(p)-Ser(p)-Ser(p) (SEQ ID NO:17); Lys-Lys-Ser(p)-Ser(p)-Ser(p)-Lys (SEQ ID NO:18); Lys-Lys-Ser(p)-Ser(p)-Ser(p)-Lys-Lys (SEQ ID NO:19); Lys-Ser(p)-Ser(p)-Ser(p)-Lys-Lys (SEQ ID NO:20); Lys-Ser(p)-Ser(p)-Ser(p)-Lys (SEQ ID NO:21); Lys-Ser(p)-Ser(p)-Ser(p)(SEQ ID NO:22); Ser(p)-Ser(p)-Ser(p)-Lys-Lys (SEQ ID NO:23); and Ser(p)-Ser(p)-Ser(p)-Lys (SEQ ID NO:24).

According to some embodiments, the formula of SEQ ID NO: 6 further comprises a blocking group (also denoted herein as a protecting group) at the C-terminus. In some embodiments, the carboxyl group at the C terminus of the peptide is protected with a protecting group. The protecting group is selected from, but not limited to an amide (i.e., the hydroxyl group at the C terminus is replaced with a primary amine (NH₂), secondary amine, or tertiary amine) or ester (i.e. the hydroxyl group at the C terminus is replaced with an ester). According to some embodiments, the blocking group is selected from the group consisting of amide and ester. According to some embodiments, the blocking group is amide.

According to some embodiments, the casein-derived peptide comprises an amino acid sequence Lys-Lys-Ser(p)-Ser(p)-Ser(p)-NH₂ (SEQ ID NO:25).

According to some embodiments, the casein-derived peptide comprises an amino acid sequence RELEELNVPGEIVES(p)LS(p)S(p)S(p)EESITRINK (SEQ ID NO:26).

The casein-derived peptide according to the invention may comprise “L” as well as “D” form residues. While the amino acid residues of the peptide sequences set forth in SEQ ID NOs:1-26 are all in the “L” isomeric form, residues in the “D” isomeric form can substitute any L-amino acid residue so long as the resulting peptide analog retains at least part of the biological activity of the corresponding “L” isomer. One reason for designing casein-derived peptides comprising at least one D-amino acid is to increase stability of the peptide to proteolytic degradation.

It should be noted that the peptides described herein are different than any known intact protein.

The therapeutic combination described herein also comprises a teat seal composition such as a composition comprising a metal salt such as a metal or heavy metal, a hydrogel, a polymer, a gel, a dip, a biofilm. The teat seal composition used herein is to be understood as a product, e.g. a composition or formulation forming a physical barrier in the teat canal of animal and thus preventing entry of microorganisms into the teat. In some embodiments, the teat seal composition comprising the metal salt formulation comprises a gel base. In some embodiments, the metal salt is in the gel and the metal salt is selected from the group consisting of bismuth, titanium, zinc, barium, and any combination thereof. The teat seal composition is of sufficiently low viscosity to facilitate application to the teat of a cow via the streak canal, such that the formulation remains sufficiently elastic to enable it to remain in place during any stage in the lactation cycle including the dry period to allow it to be optionally readily milked-out at the onset of lactation.

In some embodiments, the heavy metal salt is selected from the group consisting of bismuth sub-nitrate, titanium dioxide, zinc oxide and barium sulfate or a combination of the same.

In some embodiments, the therapeutic combination included hydrogel, polymers or gel from natural origin from mineral, vegetable or animal; or semi-synthetic or synthetic polymers or gel selected from the group, but not limited to, cellulose, wood, wool, cotton, silk, DNA, RNA, polysaccharide, chitin, chitosan, proteins, resins, polypeptides, keratin, rubber, vulcanized rubber, collagen, glycogen, paraffin, silicone.

In some embodiments, by rapidly forming the hydrogel when applied, the method of forming a teat sealant is fast, clean, “touchless” (hands free), and simple. Because it forms in situ, the sealant is highly conformal to the teat interior or exterior surface which ensures a better seal against infection and that any included active agents are more efficiently delivered directly to the teat.

Also, the invention provides such methods wherein the animal is a livestock animal, more particularly heifers or cows. Also, the invention provides such methods wherein the teat seal composition is administered during the transition period through the dry period of the heifer or cow. Also, the present invention provides such methods wherein the teat seal composition is administered to the animal by infusion into the teat of the animal or where the composition is administered to the animal by external application to the teat of the animal.

According to other embodiments, hydrogel polymeric and gel have different roles including but not limited to such as binders solubilizer, emulsifiers, paraffin, suspending gelling agent, thickeners or viscosity enhancers, bio-adhesives, thermodynamic compatibility, thermo-responsive, matrix formers, emulsifiers; also having different therapeutic activity, including but not limited to immune enhancer, biological response modifier, promotor therapeutic activity.

In some embodiments, the metal salt is present in a range of from 10 percent to 80 percent by weight, or in the range of from 30 percent to 70 percent by weight of the gel base. In accordance with the present disclosure, the therapeutic combination may be administrated to a mammalian animal in need thereof for improving a condition associated with the mammary gland in a female mammalian animal.

The present disclosure provides a method for improving a condition associated with the mammary gland of a female mammalian animal, the method comprising administration to the female mammalian animal in need thereof, a therapeutic combination comprising an amount of at least one casein-derived peptide and a teat seal composition, the teat seal composition comprising a formulation comprising a non-toxic heavy metal salt, the amount of one of the at least one casein-derived peptide and of the teat seal composition being effective to improve a condition associated with the mammary gland.

According to the principles of the present invention, and without being limited to any theory or mechanism, the combinations according to the present invention, e.g. combinations of casein proteins or casein-derived peptides and teat seal compositions, may have a beneficial effect which surpasses the beneficial effect of each component alone. Such superior beneficial effect may be supra-additive and/or synergistic, as determined by e.g. known analytical and/or statistical methods.

Improving as used herein refers for example to treating, enhancing, preventing, inhibiting, reducing, eliminating, protecting, improving welfare, improving milk quality or delaying the onset of a condition.

It was surprisingly found by the inventors that the combination described herein may be fast, clean, and easy administrated at any stage during the lactation cycle. The lactation cycle is the period between one calving and the next and includes four phases, the early, mid, and late lactation, and the dry period. The dry period is the period before parturition in which milking (lactation) is ceased and is essential to complete the process of involution, after which the milk secretion capacity is restored toward parturition.

In some embodiments, an antimicrobial or anti-inflammatory agent may be administered simultaneously or consecutively with the compositions of the present disclosure.

Thus, the present disclosure provides an advantage in management of lactating animals as it can be safely administered upon need. In some embodiments, the therapeutic combination is administrated during the lactating period. In some other embodiments, the therapeutic combination is administrated after the last milking in a lactation cycle in order to prevent development of mastitis during the dry period and around the calving period. In some other embodiments, the therapeutic combination is administrated after parturition during the next lactating period.

In some embodiments, the therapeutic combination is administrated during the late stage of the dry period, i.e. just before lactation.

In some embodiments, the therapeutic combination is administrated during the dry period (i.e. non-lactating period).

In some embodiments, the at least one casein-derived peptide and the teat seal composition may be administrated separately, i.e. in separate formulations or mixed into a single blend.

In some embodiments, the casein-derived peptide and the teat seal composition are administered in separate formulations.

In some embodiments, the at least one casein-derived peptide is administrated in combination with a pharmaceutical acceptable carrier. Thus, the present disclosure also provides a pharmaceutical composition comprising at least one casein-derived peptide. In some embodiments, the pharmaceutical composition comprising between about 1 ng/ml to about 500 mg/ml of the at least one casein-derived peptide. In some other embodiments, the at least one casein-derived peptide is in a solution, the solution comprising a pH above 6.0, at times between about 6.0 to about 11.0, at times between about 7.0 to about 9.0. In some embodiments, the pharmaceutical composition comprising the at least one casein-derived peptide is in a form of clear sterile solution, optionally substantially devoid of micelles.

The present disclosure encompasses single administration of the casein-derived peptide (or the pharmaceutical composition comprising the peptide) or multiple administrations. In some embodiments, the at least one casein-derived peptide (or the pharmaceutical composition comprising the peptide) is administered between one to ten times, at times between one to five times, at times between one to three times. In some other embodiments, the casein-derived peptide is administered at intervals of from about 1 hour, about 6 hours to about 24 hours, at times at intervals of 6 hours, at times at intervals of 8 hours, at intervals of 12 hours, at intervals of 16 hours, at intervals of 20 hours, at intervals of 24 hours or 48 hours between each administration of casein-derived peptide. In some embodiments, the at least one casein-derived peptide is administered once.

In some other embodiments, the teat seal composition is administered during or after termination of administration of the casein-derived peptide. In some further embodiments, the teat seal composition and the at least one casein-derived peptide are sequentially administrated, in at about 72 hours interval. In some other embodiments, the teat seal composition and the at least one casein-derived peptide are sequentially administrated, at about 24 hours interval. In some other embodiments, the teat seal composition is administered up to 1 hour, up to 6 hours, or up to 12 hours after termination of administration of the at least one casein-derived peptide. In some other embodiments, the teat seal composition is administered immediately after administration of the at least one casein-derived peptide.

It should be noted that if the at least one casein-derived peptide and the teat seal composition are administrated in a single formulation, the relative proportions of these ingredients in such a single formulation may be identical or different to those in a formulation which is to be applied as two separate formulations.

The formulations and compositions described herein either if administered separately or together as one blend may be adopted to any required administration route.

In some embodiments, the at least one casein-derived peptide and the teat seal composition such as sealant, hydrogel, polymer or gel formulation are formed for topical administration.

It should be understood that according to the principles of the present disclosure, the administration/application of the casein protein or casein-derived peptide composition, and the administration/application of the teat seal composition, may be independent. In certain embodiments, the casein protein or casein-derived peptide composition and the teat seal composition are administered topically. In certain embodiments, the casein protein or casein-derived peptide composition and the teat seal composition are administered internally. In certain embodiments, the casein protein or casein-derived peptide composition and the teat seal composition are administered intramammary. In certain embodiments, the casein protein or casein-derived peptide composition is administered internally, and the teat seal composition is administered topically. In certain embodiments, the casein protein or casein-derived peptide composition is administered topically, and the teat seal composition is administered internally. In other embodiments, the casein protein or casein derived peptide composition and the teat seal composition are both administered topically. In other embodiments, the casein protein or casein derived peptide composition and the teat seal composition are both administered internally.

In some embodiments, the casein-derived peptide or composition comprising the casein-derived peptide and the teat seal composition may be administered to the teat canal of an animal directly. As appreciated, in case a single formulation is used, administration into the teat canal is in one step. Alternatively, in case administration is in two separate formulations, administration is in at least two steps.

The glands and teats of domestic animals are collectively known as udder. The structure of udder of a cow is composed of two halves, each of which has two teats comprise teat surface, the teat canal and the teat cistern, and each teat drains a separate gland (quarter). In some embodiments, the at least one casein protein or casein-derived peptide and the teat seal composition are administrated to at least one gland of the mammary gland. In some further embodiments, the at least one casein protein or casein-derived peptide and the teat seal composition are administrated to at least one udder. In some further embodiments, the at least one casein protein or casein-derived peptide and the teat seal composition are administrated to the surface of one mammary gland. In some other embodiments, the at least one casein protein or casein-derived peptide and the teat seal composition are administrated into all surfaces of the animal mammary gland. In some further embodiments, the at least one casein protein or casein-derived peptide and the teat seal composition are administrated into at least one teat canal of the mammary gland. In some other embodiments, the at least one casein protein or casein-derived peptide and the teat seal composition are administrated into all teat canal of the mammary gland. In some further embodiments, the at least one casein protein or casein-derived peptide and the teat seal composition are administrated into at least one teat cistern of the mammary gland. In some other embodiments, the at least one casein protein or casein-derived peptide and the teat seal composition are administrated into all teat cisterns of the mammary gland.

As used herein “mammary gland condition” or “condition associated with the mammary gland” refers any condition, disease, involution process, disorder, welfare, milk quality, milk quantity and related symptoms being affected by the state of the mammary gland.

In some embodiments, the mammary gland condition is a mammary gland infection. Thus, the present disclosure provides in accordance with some embodiments, a method for treating, preventing, inhibiting, reducing, eliminating, protecting or delaying the onset a mammary gland infection in a female mammalian lactating animal, the method comprising administration to the animal in need thereof, a therapeutic combination comprising an amount of at least one casein protein or casein-derived peptide and a teat seal composition, the amount of which being effective in treating, preventing, inhibiting, reducing, eliminating, protecting or delaying the onset a mammary gland infection in a female mammalian lactating animal.

In some other embodiments, the mammary gland infection is mastitis. As used herein the term “mastitis” refers to an inflammation of a mammary gland or an udder, caused by a variety of causes, such as physical injury, introduction of chemicals, viruses, fungus, parasites or, most commonly, bacterial invasion and their toxins. Mastitis can be classified according two different criteria: either according to the clinical symptoms or depending on the mode of transmission (contagious mastitis or environmental mastitis). “Mastitis” is used to describe all forms of such inflammation, including subclinical and clinical mastitis, clinical mastitis including mild, pre-acute mastitis, sub-acute mastitis severe and chronic mastitis.

In subclinical mastitis, no swelling of the mammary gland or udder is detected nor is there observable abnormalities in the milk. Special screening tests, however, such as the California Mastitis Test (CMT), Wisconsin Mastitis Test (WMT) based on an estimation of somatic cell counts and the catalase test will show changes in the milk composition. This type of mastitis is commonly referred to as “hidden.” Clinical mastitis can be mild or acute and is characterized by the presence of leukocytes in the milk. Mild clinical mastitis involves changes in the milk appearance including presence of flakes or clots, watery milk or other unusual forms of the milk. Mild clinical mastitis may be accompanied by other symptoms including hot, sensitive or swollen gland or udder. Severe clinical mastitis involves the symptoms of hot, sensitive, firm gland or udder that is quite painful to the lactating animal. The onset of severe clinical mastitis is sudden, and the lactating animal may become ill showing signs of fever, rapid pulse, depression, weakness and loss of appetite. When the whole lactation system of the animal is affected, the condition is referred to as acute systemic mastitis. The severe symptoms may be also accompanied with cessation of milk production. Chronic mastitis is persistent udder infection, typically in the form of subclinical mastitis, which occasionally can develop into the clinical form and back to the subclinical form. Chronic mastitis is characterized by hard lump within the mammary gland due to the establishment of bacteria and the formation of connective tissue. In some embodiments, mastitis is clinical mastitis. In some other embodiments, mastitis is subclinical mastitis. In some further embodiments, mastitis is chronic mastitis.

In some embodiments, the mammary gland condition is cessation of milk. In some embodiments, the mammary gland condition is abrupt cessation of milk. In some embodiments, the mammary gland condition is gradual cessation of milk.

In some embodiments, the mammary gland condition is reducing length of the dry period. Thus, the present disclosure provides in accordance with some embodiments, a method for reducing the length of the dry period between cycles of lactation in a female mammalian lactating animal, the method comprising administration to the animal in need thereof, a therapeutic combination comprising an amount of at least one casein protein or casein-derived peptide and a teat seal composition, the teat seal composition comprising a heavy metal salt.

Generally, the length of dry period in non-pastoral dairy cows is between 20 to 85 days, wherein in pastoral dairy cows the length of dry period is between 30 days to 150 days. Improving the mammary gland condition according to the present embodiment, refers to a reduction in the length of the dry period for to less than about 60 days, to less than about 50 days, or to between about 20 days to about 40 days. In some other embodiments reducing the dry period length as described herein does not affect milk yield and/or milk quality.

In some other embodiments, the mammary gland condition is increasing milk yield of an animal.

Thus, the present disclosure provides in accordance with some embodiments, a method for increasing milk yield in a female mammalian lactating animal, the method comprising administration to the animal in need thereof, a therapeutic combination comprising an amount of at least one casein protein or casein-derived peptide and a teat seal composition, the teat seal composition comprising a heavy metal salt, or hydrogel or polymers, or gel, the amount of which being effective to increase milk yield. In some embodiments, the therapeutic combination is administered at the same time of cessation of milking.

Administration of the combination described herein, increase the milk yield in a lactating period subsequent to the dry period. In some embodiments, the increase in the milk yield is by about at least 0.5%, at times at least 4%, at times up to 15%, at times between 0.5% to about 15% compared to milk yield of a control cow.

In some further embodiments, the mammary gland condition is increasing milk hygiene of an animal.

Thus, the present disclosure provides in accordance with some embodiments, a method for increasing milk hygiene in a female mammalian lactating animal, the method comprising administration to the animal in need thereof, a therapeutic combination comprising an amount of at least one casein protein or casein-derived peptide and a teat seal composition, such as comprising a heavy metal salt, or hydrogel, in an amount being effective to increase milk hygiene. In some embodiments, the therapeutic combination is administered at the same time of cessation of milking.

The hygiene of the milk produced, as measured by the somatic cell counts (SCC) per ml of milk, has a great influence on the profitability of the herd, as milk comprising high levels of cells per ml of milk must be discarded. In some embodiments, the increase in milk hygiene is indicated by a reduction of the SCC in the milk as compared to the SCC before administration of the combination or in milk a cow treated with casein-derived peptide alone or with the teat seal composition alone. According to some embodiment, SCC after administration is about 20,000,000 cells per ml of milk and less, 15,000,000 cells per ml of milk and less, 10,000,000 cells per ml of milk and less, 5,000,000 cells per ml of milk and less or 1,000,000 cells per ml of milk and less. According to some embodiments, SCC after administration is about 750,000 cells per ml of milk and less, 600,000 cells/ml and less, 400,000 cells/ml and less, 300,000 cells/ml of milk, or 200,000 cells/ml of milk and less. According to some other embodiments, the SCC is reduced during the lactating cycle in which the treatment is applied. According to some further embodiments, the SCC is reduced during a lactating cycle subsequent to a dry period after the treatment is applied.

In some further embodiments, the mammary gland condition is improving milk quality. The quality of the milk may be affected by the presence of metal residues present at any teat seal composition, which in addition to being not desirable may limit the use of the milk for dairy products such cheese, e.g. mature cheese and/or affect the milking machinery. It was suggested that since the casein protein or casein-derived peptide improves the mammary gland involution process, metal residues are washed out during the process. In other words, and without being bound by theory, administration of casein protein or casein-derived peptide prior to administration of teat seal composition prevent or reduce the presence of metal in milk and related milk products. In some other embodiments, the condition associated with the mammary gland is black spot defect (BSD).

As appreciated, by treating and/or preventing at least one condition associated with the mammary gland, the treated animal has a reduction in suffering, improved comfort during the dry period or the lactation period and improved welfare. Improving the welfare may be determined, without being bound to specific conditions as reduction of steps per day and prolonging lying period per day of the animal.

As used herein, the term “livestock welfare” or “welfare in animal farm” refers to the prevention of suffering and increasing the presence of positive feelings, usually called comfort or pleasure, resulting from, inter alia, an increase lying periods, an increase in ruminating time, a decrease in metabolic need, a decrease in udder pressure and/or teat leakage, decrease in incidence of mastitis and other diseases, and decrease in lameness effect due to high milk yield.

The animal according to the present disclosure is a lactating animal. In some embodiments, the animal is a non-human being. In some embodiments, the animal is selected from the group consisting of a cow, a goat, a sheep, a buffalo, a camel, a donkey, a llama, a horse, a pig, a cat and a dog. In some embodiments, the animal is a cow. In some embodiments, the animal is a human being.

The present disclosure also provides a kit for improving, such as treating, preventing, enhancing, inhibiting, reducing, eliminating, protecting, improving welfare, increasing milk yield, or delaying the onset of a condition associated with the mammary gland in a female animal. In some embodiments, the kit comprises two parts. In some embodiments, each part is a medical device (intramammary syringes) comprises a teat seal composition, formulates as a hydrogel or a gel, and the second syringe comprises at least one casein protein or casein-derived peptide. In some embodiments, the kit contains a single or double barrel intramammary syringe comprising a blend of teat seal composition and at least one casein protein or casein-derived peptide. The kit may further comprise instructions for use.

The term “casein” as used herein generally refers to a family of related phosphoproteins (αS1, αS2, β, κ) commonly found in mammalian milk.

The term “teat seal” or “teat sealant” as used herein generally refers to any composition or formulation which forms a physical barrier when applied to a teat of a mammal animal.

The term “hydrogel” as used herein refers to crosslinked hydrophilic polymer chains.

The term “treatment” concerns improvement of at least one undesired manifestation of the disease such as increase in disease free periods, decrease in acute disease periods (in time and severely), decrease in severity of the disease, improvement in life quality, decreased mortality, decrease in the rate of disease progression as well as prophylactic treatment before disease occurs. More specifically, the term “treatment or prevention” as used herein, refers to the complete range of therapeutically positive effects of administrating to a subject including inhibition, reduction, alleviation and relief from a disorder or any related condition and illness, symptoms or undesired side effects or related disorders. It should be appreciated that the terms “inhibition”, “moderation”, “reduction” or “attenuation” as referred to herein, relate to the retardation, restraining or reduction of a process by any one of about 1% to 99.9%, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 600% to 65%, about 65% to 70%, about 75% to 80%, about 80% to 85% about 85% to 90%, about 90% to 95%, about 95% to 99%, or about 99% to 99.9%.

By “subject in need” it is meant any mammal who may be affected by the above-mentioned conditions, and to whom the treatment methods herein described is desired.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to +10%.

As used herein, the term “average molecular weight” refers to the mean plus or minus standard deviation of the molecular weight of the peptide or protein as measured by a method known to a person skilled in the art. Such methods include, for example, SDS-gel electrophoresis and size exclusion chromatography in an apparatus such as HPLC, wherein the sample is run against Standards with known molecular weight.

### EXAMPLES

### Example 1. Retention Post-Calving of Residues of Internal Teat Sealant or its Ingredients Product Inside Dairy Cow Teats

The objective of this study is to determine metal residual from internal teat sealant in freshening milk after dry cow therapy by intramammary administration of casein peptides following internal teat sealant and compared to internal teat sealant alone.

Dairy cows are evaluated between 6 f1 days before expected dry off period and are included in the study but for the following exclusion criteria: not four functional udder quarters, bacteriologic positive results from any of both pre-treatment milk samples to any microorganisms, received antimicrobial, hormone and/or anti-inflammatory therapy for any condition in the 4 weeks prior to dry-off, evidence of clinical or subclinical mastitis, cows intended for culling.

Thirty-six dairy cows are enrolled (144 udder quarter). At drying-off and after clinical examination, each udder quarter are randomly allocated to receive 1 of 4 intramammary infusions, as follows:
Group 1. Casein protein or casein-derived peptides alone. 20 ml of casein protein or casein-derived peptides are infused into the teat canal one time.
Group 2. Internal teat sealant alone is infused into the teat canal one time containing 4 g of a blend of bismuth sub-nitrate (65%) in liquid paraffin.
Group 3. Casein-derived peptides followed of internal teat sealant—20 ml of casein-derived peptides is intramammary infused and immediately followed by an infusion of 4 g of a blend of bismuth sub-nitrate (65%) in liquid paraffin.
Group 4. A group of untreated cows matched at dry off by parity will serve as control to bismuth residual detection in freshening milk.

Milk and milk secretion samples from all study groups are twice collected during the pre-drying period (Days −6±1 and after two to 3 days) for somatic cell counts, bacteriology tests and milk compositions. The second sample will be also collected to test bismuth concentration in milk.

The administration protocol was designed to ensure that each product was administered uniformly among teat physiological locations, thirty-six udder teats each per treatment arm in addition to untreated controls.

Teats are examined on day 7 and on day 14 post-treatment to detect redness, swelling and/or sealant leakage. At each interval time for sampling, clinical examination procedures and at calving the examinations will be performed in a blinded manner.

After calving, quarter milk samples from all teats are aseptically collected to identify residual bismuth concentration and cultured for intramammary infections. A 1^{st} milk sampling post-calving is taken at the first regular milking after calving following the colostrum stage (approximately up to 5 days after calving). A second 2^{nd} milk sampling post-calving 4 to 7 days after the 1′ post-calving sampling day.

### Example 2. Comparison of the Prevention of Intramammary Infection Between Casein Protein or Casein-Derived Peptides Administered Intramammary at Dry-Off Followed by Physical Barrier Devices—Internal Teat Sealant Vs. Administration of Internal Teat Sealant Alone

The objective is to determine the prevention of intramammary infection post-calving, after dry-off therapy by intramammary administration of casein protein or casein-derived peptides following internal teat sealant compared to drying off treatment with internal teat sealing alone.

### Introduction

Drying-off treatment of cow's mammary gland with casein protein or casein-derived peptides, immediately upon intra-canal administration, is expressed by enhance the influx of neutrophil cells and macrophages but also by increasing the innate immunological system which results in the prevention of intra-mammary infections, increase comfort during the non-lactating period. The use of physical barrier of such internal teat sealant alone, provides a physical barrier to invasion by broad spectrum mastitis-causing pathogens at drying-off. Consequently, udder quarters infused with bovine casein hydrolysate (Casein protein or casein-derived peptides) following by internal teat sealant would have a superior proportion of prevention from using both treatments combine at dry off comparable to each one infuses alone, post-calving.

### Study Design

A multicentered, blinded, randomised clinical field study, allocating animals without mastitis before dry-off into four treatment groups in a ratio of 1:1:1:1 (one dose of Casein protein or casein-derived peptides), internal teat sealant (ITS) alone, one dose of Bovine casein hydrolysate (bCNH) followed by ITS administration, and negative control). The study population is evenly divided between primiparous and multiparous. Up to 176 cows (44 per group); as estimated by statistical power sample size calculations and an expected dropout rate are enrolled in the study. Dairy cows are enrolled from farms using herd book platform & services (containing information of monthly SCC, calving, treatment, vaccination, fertility, and disease history data readily available). Dairy cows are not eligible for inclusion in the study if there is evidence of clinical mastitis and positive bacteriological test results, with visible teat damage, are not expected for dry-off and, have been administered within 28 days antimicrobial, hormone or anti-inflammatory drugs, vaccinated against mastitis pathogens or are difficult to control.

TABLE 1
below summarizes the study treatment

| | | | | | Number of |
|---|---|---|---|---|---|
| | | | Route of | | animals per |
| | Treat- | | adminis- | Study | treatment |
| Group | ment | Dosage | tration | day | group (up to) |
| T1 | Negative | NA | NA | 0 | 44 |
| | control | | | | |
| T2 | bCNH | 20 ml, | Intramam- | 0 | 44 |
| | | 60 mg/ml, | mary once | | |
| | | (1200 mg) | | | |
| T3 | ITS | 4 g, 5 ml | Intramam- | 0 | 44 |
| | | | mary once | | |
| T4 | bCNH | 20 ml, 60 mg/ml | Intramam | 0 | 44 |
| | following | (1200 mg) + | mary once | | |
| | ITS | ITS 5 ml (4 g) | | | |

* Each cow in the study will receive the identical treatment administered to all quarters of the udder (i.e., each cow in the study will receive either T2, or T3, or T4, or won't be treated (T1)).

This case-control is a multi-centre, blinded randomised study.

Blinded study is performed by two independent investigational groups (Lead Investigator and/or Sub-Investigator, and Treatment Administrator).

Lead Investigator and/or Sub-Investigators responsible for making clinical examination and observations, adverse event assessments, medical decisions of any sort, sampling and filling the clinical research form.

The Random Treatment Allocation Plan (RTAP), randomisation table and inventory records will be visible only to Treatment Administrator and to the study clinical research coordinator, who is responsible to assign the animals into the randomisation table and manage the treatment investigational product inventory.

Treatment day is defined as Day 0 which it the dry treatment day (dry-off date).

Before treatment, the teats should be thoroughly cleaned and disinfected before each infusion (for all treatment groups, including negative control cows), and care should be taken to avoid contamination of the injection nozzle.

### Assessment of Efficacy

The parameters used to assess the efficacy of the bCNH and ITS are listed in the following table 2:

| Parameter | Timing | Method | Procedures |
|---|---|---|---|
| 1. Udder examination | Day (−6) ± 1 | Udder: | Cattle Physical |
| (by observation and | 1 to 3 days later | Confirmation, | Examination Guideline |
| palpation) | Day 0 | symmetry, | |
| | Day 7 ± 2 and | consistency, | |
| | 14 ± 2 post- | injury, pain, | |
| | treatment | redness and | |
| | (without milk | temperature | |
| | appearance) | Milk appearance: Color | |
| | Up to day 5 | and consistency | |
| | post-calving (on | | |
| | the same day of | | |
| | the 1^{st} milk | | |
| | sampling) | | |
| | 4 to7 days later | | |
| 2. Milk culture | 1^{st} pre-treatment | Internal SOPs based on | Bacteriology |
| microbiology results | milk sampling - | US NMC Laboratory | analyses will be |
| | Day (−6) ± 1 | Handbook, 2017 * | performed by the |
| | 2^{nd} pre- | | Laboratory for |
| | treatment milk | | Udder Health & |
| | sampling 1 to 3 | | Milk Quality. |
| | days after the 1^{st} | | |
| | sampling | | |
| | 1^{st} milk | | |
| | sampling post- | | |
| | calving- taken | | |
| | before the first | | |
| | regular milking | | |
| | after calving | | |
| | following the | | |
| | colostrum stage | | |
| | (approximately | | |
| | up to 5 days | | |
| | after calving) | | |
| | 2^{nd} milk sampling post- | | |
| | calving 4 to 7 days after | | |
| | the 1^{st} post-calving | | |
| | sampling day | | |

* National Mastitis Council, 2017. Laboratory handbook on bovine mastitis. National Mastitis Council (NMC), 126 West Main Street, New Prague, MN 56071 USA, incorporated herein by reference.

### Assessment of Safety

Safety examination, observations and measurements are listed in the following table 3:

| Parameter | Timing | Method | Procedures |
|---|---|---|---|
| 1. Physical examination | Day (−6) ± 1 | A physical | Cattle Physical |
| | Day 0 | examination of the | Examination Guideline |
| | Day 7 ± 2 and 14 ± 2 | cow will include | |
| | post-treatment | rectal temperature, | |
| | On the same day of the | pulse, respiratory | |
| | 2^{nd} post-calving | rate. | |
| | sampling (after the first | A general clinical | |
| | post-calving | examination of the | |
| | bacteriology sampling) | following systems will | |
| | | include Respiratory, | |
| | | Cardiovascular, | |
| | | Gastrointestinal, | |
| | | Urogenital, | |
| | | Musculoskeletal and | |
| | | Feet, Integument, | |
| | | Sensory Nervous | |
| | | system, Lymphatic | |
| | | system, Ocular system, | |
| | | Behaviour, Udder. | |
| 2. Udder examination | Day (−6) ± 1 | Local reaction | Cattle Physical |
| | 1 to 3 days later | measurements: | Examination Guideline |
| | Day 0 | conformation, | |
| | Day 7 ± 2 and 14 ± 2 | symmetry, | |
| | post-treatment | consistency, injury, | |
| | (without milk | pain, redness and | |
| | appearance) | temperature will be | |
| | On the same day of | evaluated. | |
| | the 1^{st} post-calving | Milk appearance: color | |
| | sampling day | and consistency | |
| | 4 to 7 days later (on the | | |
| | same day of the 2^{nd} | | |
| | post-calving | | |
| | bacteriology sampling) | | |
| 3. Calving information | Upon calving (the data | Post calving ease score | Cattle Physical |
| and calf examination | will be recorded in the | from NOA or Farm | Examination Guideline |
| | NOA by the farm | manager testimony. | |
| | manager and then | Calf Physical | |
| | recorded in the CRF | Examination, | |
| | soon after calving). | | |
| | Up to day 2 post- | | |
| | calving. | | |

### Assessment of Other Parameters

Other measured parameters are listed in the following table 4:

| Parameter | Timing | Method | Procedures |
|---|---|---|---|
| 1. Somatic | Day (−6) ± 1 | Method based on ISO | SCC |
| cell counts | On the | 13366-2 IDF 148-2 | analyses are |
| | same day | | performed by |
| | of the 2^{nd} | | the ICBA |
| | sampling | | Central Milk |
| | post- | | Laboratory |
| | calving | | |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A therapeutic combination, comprising:
(i) a composition comprising at least one casein protein or casein-derived peptide; and
(ii) a teat seal composition.

2. The combination of claim 1, wherein the at least one casein protein comprises at least one of β-casein, αS1-casein, αS2-casein, or κ-casein.

3. The combination of claim 1, wherein the at least one casein-derived peptide comprises at least one fragment of β-casein, αS1-casein, αS2-casein, or κ-casein.

4. The combination of claim 1, wherein the at least one casein-derived peptide comprises a casein hydrolysate.

5. The combination of claim 1, wherein the at least one casein-derived peptide comprises a phosphopeptide.

6. The combination of claim 5, wherein the phosphopeptide comprises an amino acid sequence selected from the group consisting of SEQ ID NO:1 to SEQ ID NO:26.

7. The combination of claim 1, wherein the teat seal composition forms a physical barrier on a teat surface, in a teat canal, or in a teat cistern of an animal.

8. The combination of claim 1, wherein the teat seal composition is formulated as a dip, a hydrogel, or a biofilm.

9. The combination of claim 1, wherein the teat seal composition comprises a heavy metal, or salt thereof.

10. The combination of claim 9, comprising a non-toxic heavy metal salt.

11. The combination of claim 10, wherein the non-toxic heavy metal salt is selected from the group consisting of bismuth salt, titanium salt, zinc salt, barium salt, and any combination thereof.

12. The combination of claim 1, wherein the teat seal composition comprises 40% to 80% by weight heavy metal salt.

13. The combination of claim 1, wherein the casein-derived peptide composition comprises between 10 ng/ml to 500 mg/ml of the at least one casein-derived peptide.

14. The combination of claim 1, wherein the at least one casein protein or casein-derived peptide composition and the teat seal composition are comprised in the same composition.

15. The combination of claim 1, wherein the at least one casein protein or casein-derived peptide composition and the teat seal composition are comprised in different compositions.

16. The combination of claim 15, wherein the at least one casein protein or casein-derived peptide composition and the teat seal composition are administrated separately.

17. The combination of claim 15, wherein the at least one casein protein or casein-derived peptide composition and the teat seal composition are administrated together.

18. The combination of claim 1, wherein the at least one casein protein or casein-derived peptide composition or the teat seal composition further comprise a pharmaceutical carrier.

19. The combination of claim 1, wherein the pH of the at least one casein protein or casein-derived peptide composition or the teat seal composition is in the range of between about 6.5 to about 9.0.

20. A method for:
(a) treating, preventing, inhibiting, reducing, eliminating, protecting or delaying the onset of a mammary gland disorder;
(b) reducing the length of the dry period;
(c) increasing the milk yield;
(d) increasing the milk hygiene;
(e) increasing the cow's comfort;
(f) improving the livestock welfare;
(g) enhancing involution;
(h) increasing the rate of involution; or
(i) any combination of (a), (b), (c), (d), (e), (f) (g), and (h);
in a female mammalian animal in need thereof, the method comprising administering a therapeutic combination, comprising (i) a composition comprising at least one casein protein or at least one casein-derived peptide; and (ii) a teat seal composition; to the female mammalian animal.

21. The method of claim 20, wherein the therapeutic combination is administrated at the lactating period of the female mammalian animal.

22. The method of claim 20, wherein the therapeutic combination is administrated at the dry period of the female mammalian animal.

23. The method of claim 20, wherein the at least one casein protein or the at least one casein-derived peptide composition is administered one to six times.

24. The method of claim 20, wherein the at least one casein protein or the at least one casein-derived peptide composition is administered at intervals of from about 1 hour to about 24 hours.

25. The method of claim 20, wherein the teat seal composition is administered during the administration of the at least one casein protein or the at least one casein-derived peptide composition.

26. The method of claim 20, wherein the teat seal composition is administered after the administration of the at least one casein protein or the at least one casein-derived peptide composition.

27. The method of claim 20, wherein the teat seal composition and the at least one casein protein or the at least one casein-derived peptide composition are sequentially administrated at about 1 to about 24 hours intervals.

28. The method of claim 20, wherein the teat seal composition and the at least one casein protein or the at least one casein-derived peptide composition are sequentially administrated at about 1 to about 48 hours intervals.

29. The method of claim 20, wherein the teat seal composition and the at least one casein protein or the at least one casein-derived peptide composition are sequentially administrated at about 72 hours intervals.

30. The method of claim 20, wherein the teat seal composition is administered up to 12 hours after the administration of the at least one casein protein or the at least one casein-derived peptide composition.

31. The method of claim 20, wherein the at least one casein protein or the at least one casein-derived peptide composition and the teat seal composition are administrated to at least one mammary gland of the female mammalian animal.

32. The method of claim 20, wherein the at least one casein protein or the at least one casein-derived peptide composition and the teat seal composition are administrated to at least one udder of the female mammalian animal.

33. The method of claim 20, wherein the at least one casein protein or the at least one casein-derived peptide composition and the teat seal composition are administrated to at least one teat canal of the female mammalian animal.

34. The method of claim 20, wherein the mammary gland disorder is a mammary gland infection.

35. The method of claim 34, wherein the mammary gland infection is mastitis.

36. The method of claim 35, wherein the mastitis is selected from the group consisting of clinical mastitis, subclinical mastitis, and chronic mastitis.

37. The method of claim 20, wherein increasing the milk hygiene comprises preventing black spot defect (BSD) in cheese.

38. The method of claim 20, wherein increasing the milk hygiene comprises reducing somatic cell counts (SCC) in milk.

39. The method of claim 20, wherein the female mammalian animal is a lactating animal.

40. The method of claim 20, wherein the female mammalian animal is a non-lactating animal.

41. The method of claim 20, wherein the female mammalian animal is selected from the group consisting of a cow, a goat, a sheep, a buffalo, a camel, a donkey, a llama, a horse, a pig, a cat, and a dog.

42. The method of claim 41, wherein the female mammalian animal is a cow.

43. The method of claim 20, comprising administering between 10 ng to 200 mg of the at least one casein protein or casein-derived peptide per kg-body-weight of the female mammalian animal.

44. A kit comprising (i) a composition comprising at least one casein protein or at least one casein-derived peptide, (ii) a teat seal composition, and optionally (iii) instructions for use of the kit in a method for:
(a) treating, preventing, inhibiting, reducing, eliminating, protecting or delaying the onset of a mammary gland disorder;
(b) reducing the length of the dry period;
(c) increasing the milk yield;
(d) increasing the milk hygiene;
(e) enhancing involution;
(f) increasing the rate of involution;
(g) increasing the cow's comfort
(h) improving the livestock welfare; or
(i) any combination of (a), (b), (c), (d) (e), (f), (g) and (h).

45. The kit of claim 44, comprising at least two separate containers, first container comprising the composition comprising the at least one casein protein or at least one casein-derived peptide, and the second container comprising the teat seal composition.

46. The kit of claim 45, wherein the first container or the second container are a syringe.

47. The kit of claim 44, comprising at least one container, the container comprising both the composition comprising the at least one casein protein or at least one casein-derived peptide and the teat seal composition.

48. The kit of claim 47, wherein the container is a syringe.


SEQUENCE LISTING (26 sequences)

SEQ ID NO: 1 - protein, 4 aa, Unknown
  Description of Unknown Bovine sequence
  MOD_RES (1)..(3): May or may not be phosphorylated
SSSE

SEQ ID NO: 2 - protein, 5 aa, Unknown
  Description of Unknown Bovine sequence
  MOD_RES (1)..(3): May or may not be phosphorylated
SSSEE

SEQ ID NO: 3 - protein, 5 aa, Unknown
  Description of Unknown Bovine sequence
  MOD_RES (1)..(1): phosphorylated serine
  MOD_RES (2)..(2): phosphorylated serine
  MOD_RES (3)..(3): phosphorylated serine
SSSEE

SEQ ID NO: 4 - protein, 25 aa, Unknown
  Description of Unknown Bovine sequence
  MOD_RES (15)..(15): phosphorylated serine
  MOD_RES (17)..(17): phosphorylated serine
  MOD_RES (18)..(18): phosphorylated serine
  MOD_RES (19)..(19): phosphorylated serine
RELEELNVPGEIVESLSSSEESITR

SEQ ID NO: 5 - protein, 21 aa, Unknown
  Description of Unknown Bovine sequence
  MOD_RES (8)..(8): phosphorylated serine
  MOD_RES (9)..(9): phosphorylated serine
  MOD_RES (10)..(10): phosphorylated serine
QMEAESISSSEEIVPDSVEQK

SEQ ID NO: 6 - protein, 21 aa, Unknown
  Description of Unknown Bovine sequence
  MOD_RES (8)..(8): phosphorylated serine
  MOD_RES (9)..(9): phosphorylated serine
  MOD_RES (10)..(10): phosphorylated serine
KNTMEHVSSSEESIISNETYK

SEQ ID NO: 7 - protein, 17 aa, Unknown
  Description of Unknown Bovine sequence
  MOD_RES (11)..(11): phosphorylated serine
  MOD_RES (13)..(13): phosphorylated serine
KVNELSKNIGSESTEDQ

SEQ ID NO: 8 - protein, 20 aa, Unknown
  Description of Unknown Bovine sequence
  MOD_RES (9)..(9): phosphorylated serine
  MOD_RES (11)..(11): phosphorylated serine
PTLNREQLSTSEENSKKTVD

SEQ ID NO: 9 - protein, 24 aa, Unknown
  Description of Unknown Bovine sequence
  MOD_RES (14)..(14): phosphorylated serine
  MOD_RES (16)..(16): phosphorylated serine
  MOD_RES (17)..(17): phosphorylated serine
  MOD_RES (18)..(18): phosphorylated serine
ELEELNVPGEIVESLSSSEESITR

SEQ ID NO: 10 - protein, 24 aa, Unknown
  Description of Unknown Bovine sequence
  MOD_RES (14)..(14): phosphorylated serine
  MOD_RES (16)..(16): phosphorylated serine
  MOD_RES (17)..(17): phosphorylated serine
  MOD_RES (18)..(18): phosphorylated serine
RELEELNVPGEIESLSSSEESITR

SEQ ID NO: 11 - protein, 21 aa, Unknown
  Description of Unknown Bovine sequence
  MOD_RES (6)..(6): phosphorylated serine
  MOD_RES (8)..(8): phosphorylated serine
  MOD_RES (9)..(9): phosphorylated serine
  MOD_RES (10)..(10): phosphorylated serine
  MOD_RES (17)..(17): phosphorylated serine
QMEAESISSSEEIVPNSVEQK

SEQ ID NO: 12 - protein, 21 aa, Unknown
  Description of Unknown Bovine sequence
  MOD_RES (8)..(8): phosphorylated serine
  MOD_RES (9)..(9): phosphorylated serine
  MOD_RES (10)..(10): phosphorylated serine
  MOD_RES (16)..(16): phosphorylated serine
KNTMEHVSSSEESIISQETYK

SEQ ID NO: 13 - protein, 17 aa, Unknown
  Description of Unknown Bovine sequence
  MOD_RES (11)..(11): phosphorylated serine
  MOD_RES (13)..(13): phosphorylated serine
KVNELSKDIGSESTEDQ

SEQ ID NO: 14 - protein, 129 aa, Unknown
  Description of Unknown Bovine sequence
  MOD_RES (5)..(5): phosphorylated serine
  MOD_RES (45)..(45): phosphorylated serine
  MOD_RES (46)..(46): phosphorylated serine
  MOD_RES (47)..(47): phosphorylated serine
  MOD_RES (50)..(50): phosphorylated serine
  MOD_RES (118)..(118): phosphorylated serine
  MOD_RES (120)..(120): phosphorylated serine
ESIISQETYKQEKNMAINPSKENLCSTFCKEVVRNANEEETSIGSSSEESAEVATEEVKITVDDKHYQKALNEINQFYQKFPGYLQYLYQGPIVLNPWNQVLRNAVPITPTLNREQLSTSEENSKKTVN

SEQ ID NO: 15 - protein, 23 aa, Unknown
  Description of Unknown Bovine sequence
  MOD_RES (13)..(13): phosphorylated serine
  MOD_RES (15)..(15): phosphorylated serine
  MOD_RES (16)..(16): phosphorylated serine
  MOD_RES (17)..(17): phosphorylated serine
ELEELNVPGEIESLSSSEESITR

SEQ ID NO: 16 - protein, 7 aa, Unknown
  Description of Unknown Bovine sequence
  MOD_RES (1)..(2): A positively charged amino acid or absent
  SITE (1)..(2): This region may encompass 0-2 residues
  MOD_RES (3)..(3): phosphorylated serine
  MOD_RES (4)..(4): phosphorylated serine
  MOD_RES (5)..(5): phosphorylated serine
  MOD_RES (6)..(7): A positively charged amino acid or absent
  MOD_RES (6)..(7): This region may encompass 0-2 residues
XXSSSXX

SEQ ID NO: 17 - protein, 5 aa, Unknown
  Description of Unknown Bovine sequence
  MOD_RES (3)..(3): phosphorylated serine
  MOD_RES (4)..(4): phosphorylated serine
  MOD_RES (5)..(5): phosphorylated serine
KKSSS

SEQ ID NO: 18 - protein, 6 aa, Unknown
  Description of Unknown Bovine sequence
  MOD_RES (3)..(3): phosphorylated serine
  MOD_RES (4)..(4): phosphorylated serine
  MOD_RES (5)..(5): phosphorylated serine
KKSSSK

SEQ ID NO: 19 - protein, 7 aa, Unknown
  Description of Unknown Bovine sequence
  MOD_RES (3)..(3): phosphorylated serine
  MOD_RES (4)..(4): phosphorylated serine
  MOD_RES (5)..(5)
KKSSSKK

SEQ ID NO: 20 - protein, 6 aa, Unknown
  Description of Unknown Bovine sequence
  MOD_RES (2)..(2): phosphorylated serine
  MOD_RES (3)..(3): phosphorylated serine
  MOD_RES (4)..(4): phosphorylated serine
KSSSKK

SEQ ID NO: 21 - protein, 5 aa, Unknown
  Description of Unknown Bovine sequence
  MOD_RES (2)..(2): phosphorylated serine
  MOD_RES (3)..(3): phosphorylated serine
  MOD_RES (4)..(4): phosphorylated serine
KSSSK

SEQ ID NO: 22 - protein, 4 aa, Unknown
  Description of Unknown Bovine sequence
  MOD_RES (2)..(2): phosphorylated serine
  MOD_RES (3)..(3): phosphorylated serine
  MOD_RES (4)..(4): phosphorylated serine
KSSS

SEQ ID NO: 23 - protein, 5 aa, Unknown
  Description of Unknown Bovine sequence
  MOD_RES (1)..(1): phosphorylated serine
  MOD_RES (2)..(2): phosphorylated serine
  MOD_RES (3)..(3): phosphorylated serine
SSSKK

SEQ ID NO: 24 - protein, 4 aa, Unknown
  Description of Unknown Bovine sequence
  MOD_RES (1)..(1): phosphorylated serine
  MOD_RES (2)..(2): phosphorylated serine
  MOD_RES (3)..(3): phosphorylated serine
SSSK

SEQ ID NO: 25 - protein, 5 aa, Unknown
  Description of Unknown Bovine sequence
  MOD_RES (3)..(3): phosphorylated serine
  MOD_RES (4)..(4): phosphorylated serine
  MOD_RES (5)..(5): phosphorylated serine
KKSSS

SEQ ID NO: 26 - protein, 28 aa, Unknown
  Description of Unknown Bovine sequence
  MOD_RES (15)..(15): phosphorylated serine
  MOD_RES (17)..(17): phosphorylated serine
  MOD_RES (18)..(18): phosphorylated serine
  MOD_RES (19)..(19): phosphorylated serine
RELEELNVPGEIVESLSSSEESITRINK